(19) United States
(12) Patent Application Publication
(10) Pub. No.: US 2023/0364376 A1
(43) Pub. Date: Nov. 16, 2023

(54) APPARATUS FOR ADMINISTERING BILATERAL INDIRECT CUTANEOUS AND VISUAL STIMULATION TO A HUMAN SUBJECT (WHISPERS)

(71) Applicants: Sibylle M. Georgianna, Dana Point, CA (US); Thomas D. Georgianna, III, Dana Point, CA (US)

(72) Inventors: Sibylle M. Georgianna, Dana Point, CA (US); Thomas D. Georgianna, III, Dana Point, CA (US)

(21) Appl. No.: 17/803,178

(22) Filed: Feb. 1, 2022

(51) Int. Cl. A61M 21/00

(52) U.S. Cl. CPC A61M 21/00; A61M 2021/0022

(57) ABSTRACT

This invention generally provides two cutaneous and visual stimulation modules, or “units”. One unit is worn by a person on each wrist. Alternatively, a unit is secured to other parts of the body on opposite sides. Each unit is powered by a rechargeable battery, and contains both a cutaneous stimulation applicator, such as an electric ducted fan, and/or an analog visual stimulation applicator, such as a light/dark windowed roller. Each pair of units will synchronize with the other. Each unit includes at least one control button and a status indicator. Each unit contains a micro-controller that performs tasks such as communication, motor activation, user monitoring and control, battery monitoring, low-power sleep, and algorithm execution.

### CROSS-REFERENCE TO RELATED APPLICATIONS/U.S. PATENT DOCUMENTS: 62/501,736

### Current U.S. Class: Current CPC Class

A61M 21/02 20130101; A61M 2209/088 20130101; A61M 2205/3584 20130101; A61M 2205/3592 20130101; A61M 2230/04 20130101; A61M 2230/65 20130101; A61M 2230/205 20130101; A61M 2230/42 20130101; A61M 2230/432 20130101; A61M 2230/50 20130101; A61M 2230/60 20130101; A61M 2230/10 20130101; A61M 2205/3553 20130101; A61M 2205/505 20130101; A61M 2205/583 20130101; A61M 2205/8206 20130101; G16H 40/63 20180101; G16H 40/67 20180101; G16H 50/20 20180101; A61M 2021/0022 20130101; A61M 21/02 20060101 A61M021/02

### STATEMENT REGARDING FEDERALLY SPONSORED RESEARCH OR DEVELOPMENT

The research to this design has not received federal funds.

### BACKGROUND OF THE INVENTION

### Field of the Invention

This invention relates, generally, to apparatuses for providing bilateral stimulation to human subjects as part of psychotherapy treatment regimen and devices for administering bilateral stimulation to a human subject in order to reduce stress or anxiety experienced by the subject.

### Description of the Prior Art

In 1987, the effect of eye movement on the reduction of distressing thoughts was first discovered by Dr. Francine Shapiro during her Graduate studies. That is, first through self-observation, Dr. Shapiro noticed that moving her eyes from the lefts side of the visual field to the right side of the visual field and then back to the left side of the visual field with several repetitions seemed to decrease the intensity of disturbing thoughts experienced at the time. Dr. Shapiro published a follow up scientific study of eye movement in victims of trauma in the 1989 issue of the Journal of Traumatic Stress. The therapy treating victims of trauma via the use of eye movement was called Eye Movement Desensitization and Reprocessing (EMDR). EMDR therapists use scientifically evaluated verbal protocols that allow the therapist and the client to activate a disturbing memory and subsequently the client's body to reprocess the event by integrating it into healthy (adaptive) memories. Multiple studies by EMDRIA (the EMDR International Association) researchers and practitioners provided ample evidence that EMDR therapy is efficacious for the reduction of posttraumatic stress disorder. Other conditions that can be treated effectively include other anxiety disorders (e.g., panic disorder, phobias) sexual and/or physical abuse, work and other stress responses, pain conditions, dissociative disorders, and addictions.

To become an EMDR therapist, therapists learn to create the bilateral stimulation of the clients' brains via eye movement. That is, the therapist guides the client in moving his/her eyes from the left side of the left visual field to the right side of the right visual field, about 26 repetitions as part of one processing set. In some situations, eye movement is not the preferred method of bilateral stimulation. For example, if clients have visual impairments, struggle with dizziness or vertigo or lack the ability to remain focused on moving the eyes in the aforementioned fashion, other forms of bilateral stimulation may be indicated. Alternative forms include tactile bilateral stimulation or bilateral tones. Alternative forms may also alleviate strain on the therapist's upper body and/or fatigue when applying EMDR therapy via eye movement.

While many therapists move their fingers backwards and forwards to guide the clients' eye movement for reprocessing, devices have been developed to replace the therapist's physical involvement of bilateral stimulation in their work. On Aug. 30, 1994, U.S. Pat. No. 5,343,261 was issued to David L. Wilson for a device that created the so called saccadic eye movements. The device also included an option for utilizing bilateral tones. On Dec. 14, 1999, Jurgen G. Schmidt and Shirley Jean Schmidt received U.S. Pat. No. 6,001,073 for a device that generated alternating tactile stimulations. In an effort to reduce strain the shoulder of a person who administers the eye movement by moving his/her fingers in front of the client's face, therapists have also used sticks to facilitate the bilateral eye movements for clients: clients were asked to watch the tip of the stick moving back and forth in front of the clients' eyes.

In 1998, during Hurricane Pauline in Acapulco, Mexico, Lucina Artigas developed the so called “butterfly hug” method of bilateral stimulation during her work with the survivors of the hurricane. Clients are asked to do the butterfly hug during the reprocessing phases 4 to 6 instead of the therapist administering the bilateral stimulation via eye movement, auditory stimulation, or tapping of the person's hands or knees. In this instance, clients fold their arms across their torsos and administer alternating taps on their own, shoulders to create bilateral stimulation. Artigas and Ignacio Jarero have utilized this method worldwide during their work with survivors of man-made and natural catastrophes.

In her book “Getting Past Your Past”, Dr. Francine Shapiro describes how to use bilateral stimulation as a so called resource for strengthening good coping during challenging situations. She describes two kinds of bilateral stimulation. One kind of stimulation is created by “[. . . ] just putting your hands on your thighs and tapping first one and then the other. If bilateral stimulation helps, then use it daily.” A second kind of stimulation is created via the aforementioned butterfly hug. “As long as you were able to use it successfully, you can also try it when you're feeling stressed or anxious.” If negative thoughts and memories begin to emerge through the use of bilateral stimulation, the reader should use other self-control techniques, such as breathing techniques.

In the last couple of years, multiple software programs, applications, and other devices (e.g., for bilateral eye movement as well as for administering tones and/or music in a bilateral fashion to the consumer's auditory system) have become available to the consumer. Utilizing visual, auditory, and/or tactile bilateral stimulation, consumers can now reduce stress and anxiety themselves.

In 2016, Dr. Amy Serin and Vicki Mayo (VMAS Solutions LLC) jointly developed a handheld and wearable tactile stimulation device (formerly called “Buzzies”, then renamed to “Touchpoint”. The product consists of pair of bilateral stimulation devices that are to be worn around the two wrists. The devices provide tactile stimulation to both left and right sides of the body in an alternating fashion.

As the consumer gets to choose between means of bilateral stimulation in order to reduce stress and enhance coping during challenging times, the aforementioned three modalities_of bilateral stimulation (that is, visual through eye movement, tactile through tapping, and auditory through tones) are complementary. Someone might have a hearing impairment. Age and/or disability may keep others from manually performing the bilateral tapping. Yet, fatigue and/or distractions as well as developmentally challenged individuals may keep the consumer from sustaining the bilateral tapping for an extended period of time. Such extended times of bilateral stimulation (i.e., 26 “sets” of alternating stimulation) are necessary in order for disturbing to be effectively processed.

Gentle human touch has been associated with soothing, care, and has been found to decrease anxiety. One company developed a wireless electro-mechanical product that replicates bilateral tapping and closely mimics the sensation of human touch. The device was designed to automatically adjust the stimulation parameters without the need for continual manual intervention from either the user or the therapist. Last but not least, usage patterns were recorded so that the user and/or therapist could make data-driven changes, if desired.

### SUMMARY

Previous inventions creating bilateral stimulation through motorized pulses have been referred to as “tappers.” However, since the mechanism is different, the units described herein shall be referred to as “whispers,” or when referring to visual stimulae, “glimmers.” One of a pair of whispers will be attached to the left side of the body (with a band, in a pocket, or simply held in the hand). The other whisper will be attached to the right side of the body in like fashion. The pair will provide alternating, left/right physical stimulation.

A rechargeable battery powers each whisper. Each whisper employs a ducted fan to create a physical sensation on the skin. Each whisper will also utilize a wireless communication protocol to synchronize with the other. Each whisper will have a user interface consisting of a display and input switches to control the variables associated with the stimulae. Each whisper will have a detachable band for attaching it to the wrist, arm, or other location. Each whisper will employ a micro-controller to handle communication with the other unit, operate the EDF, read user input, manage power consumption and recharging, etc. Each whisper also utilizes a rechargeable battery that may be recharged with a charging port. The present implementation of the wireless communication protocol is proprietary.

The whispers will also have a “sleep” mode which allows a user to activate a timer that will turn the whispers off after a set amount of time. This will allow the subject to use the whispers to aid falling asleep without leaving them on all night.

In contrast to other bilateral stimulation devices which rely on either a mechanical weight being rotated on a shaft to cause a sensation or a light source to provide visual stimulation, the units here are produce cutaneous stimulation by the creation by a fan of a pressure differential in a duct that conveys the sensation of torque, or in the event of visual stimulation, the units here use an analog display that does not use electricity to emit a light source. In the case of cutaneous stimulation, the sensation of torque can be used to apply a different (i.e., gentler) stimulation profile than is available through traditional mechanical processes using a wight rotating on a shaft. In the case of visual stimulation, the analog roller creates visual stimulation without emitting a light source, which the literature on point suggests in certain instances has the potential to interfere with the sleep cycle. The preferred embodiment of the invention is comprised of a pair of communicating units, each equipped with a stepper motor driving a fan on a shaft within a duct. The sensation of torque is created by rotating the fan and accelerating the column of air within the duct. When the column of accelerated air comes into contact with the skin it will tend to stay in motion, in accordance with Newton's laws, and as its passes between the user's skin and the exit of the duct, create a pressure differential in accordance with the principles described by Bernoulli. The deceleration of the column of air as it comes into contact with the skin of the subject is perceived as pressure. In an alternative embodiment, the air column accelerates when coming into contact with the skin of the user to escape the duct, thus creating negative pressure at the base of the air column. In both cases, it is not the unit itself, but the air that the subject feels as touch. The profile of the cutaneous stimulation can be controlled by the acceleration, deceleration, and power applied to the fan. This allows the stimulation to have any variation of strong, weak, gradual, or sharp attack, sustain, and fade. The analog visual stimulation device attached to each unit may be toggled to the ducted fan, turned off, or used alone. The analog visual stimulation device works by alternately displaying white and black hemispheres of a roller which is turned by a stepper motor. Unit one will display black while unit two displays white and vice versa. The frequency of the rotation can be set by the subject such that the user's eyes are directed from the left to right and vice versa at a comfortable rate. This constitutes bilateral visual stimulation.

### Breeze Sensation

In another embodiment of the invention, the ducted fan from each unit will apply an air current against the skin of the subject creating a sensation not of pressure or a tap, but of airflow (“wind”). Again, sensation of airflow can be used to apply a different (i.e., gentler) stimulation profile than is available through traditional mechanical processes using a wight rotating on a shaft. As with the preferred embodiment, the alternate embodiment of the invention is comprised of a pair of communicating units, each equipped with a stepper motor driving a fan on a shaft within a duct. The sensation of airflow is created by rotating the fan and accelerating the column of air within the duct. When the column of accelerated air comes into contact with, and flows over the skin it will create the sensation of airflow. In this case also, it is not the unit itself, but the air that the subject. feels. The profile of the cutaneous stimulation can be controlled by the acceleration, deceleration, and power applied to the fan. This allows the stimulation to have any variation of strong, weak, gradual, or sharp attack, sustain, and fade. The analog visual stimulation device attached to each unit may be toggled to the ducted fan, turned off, or used alone.

Unlike other technology, the present invention will use encrypted communications between units and will not store or export data. Accordingly the invention will be compliant with all privacy laws and regulations (including, but not limited to, the Health Insurance Portability and Accounting Act), and moreover, be secure.

### BRIEF DESCRIPTION OF THE DRAWINGS

### Description of the Figure(s) of the Drawing

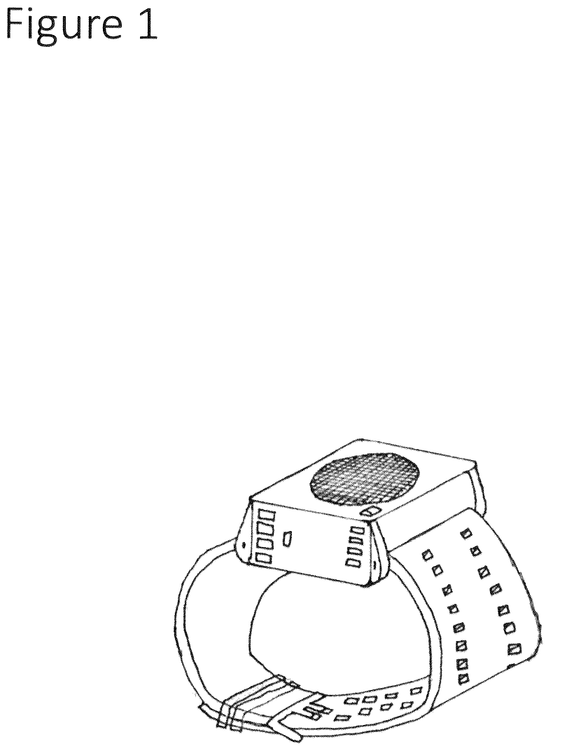

FIG. 1 is an isometric view from an elevated vantage point of a whisper;

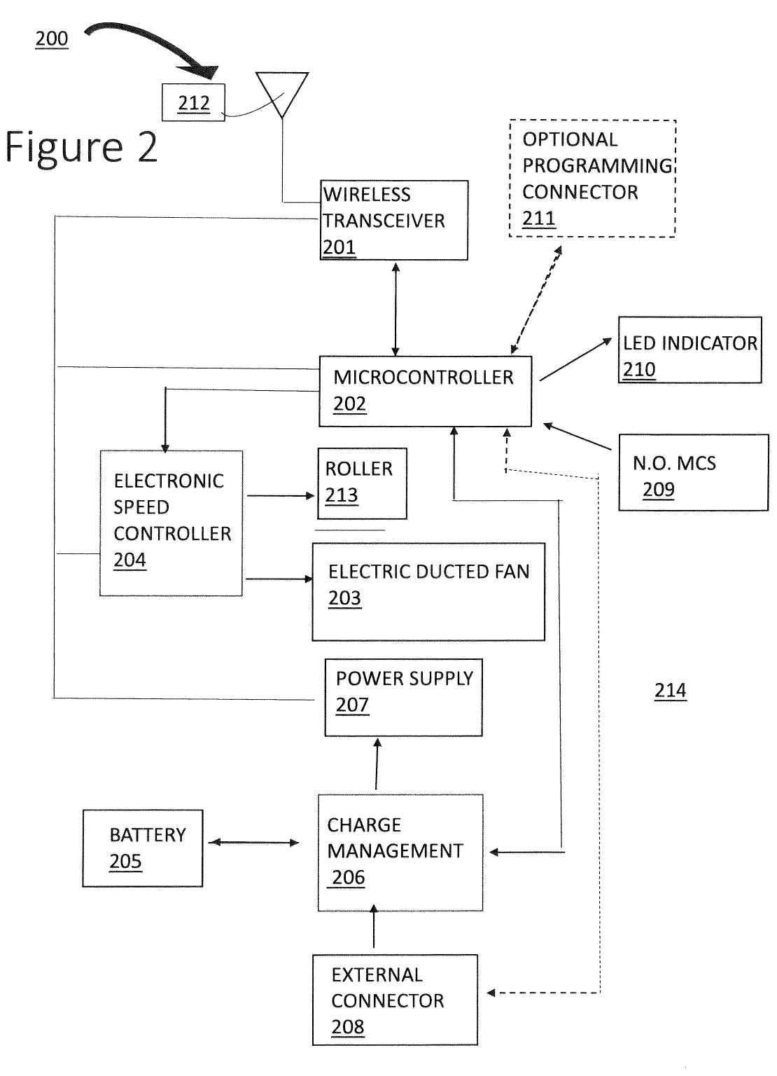

FIG. 2 Is a block diagram of a whisper;

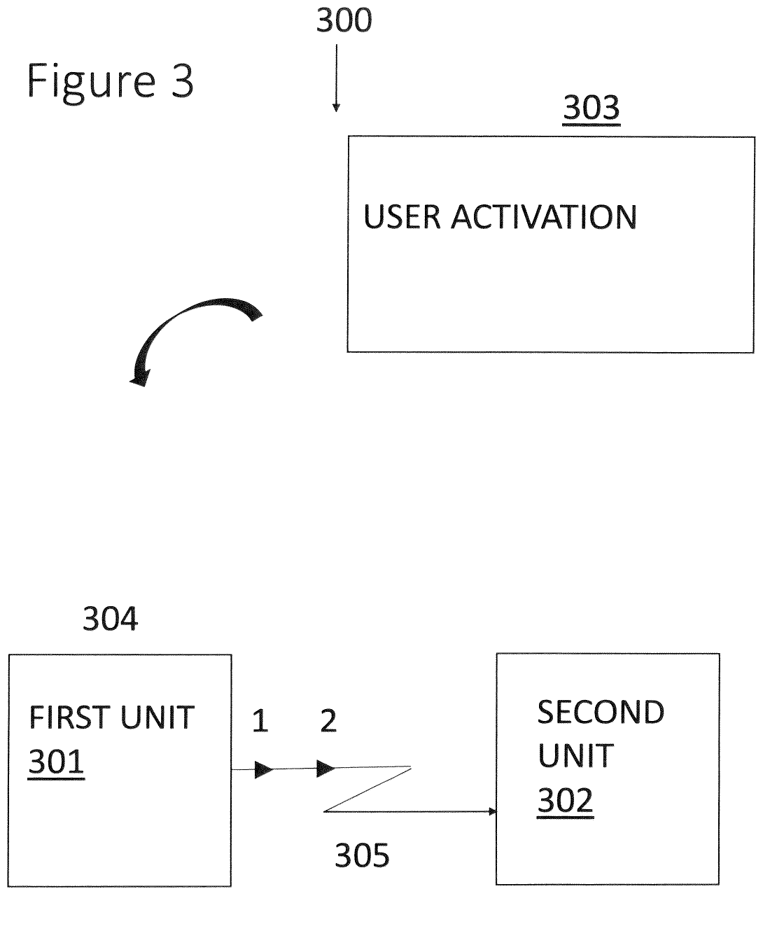

FIG. 3 is a block diagram of a pair of whispers including the first and second units which are wirelessly connected to each other, and user activation in which the communication protocol between the first and the second whisper includes two separate “handshakes” initiated by the first whisper that include information on duration and intensity of the pulse, respectively;

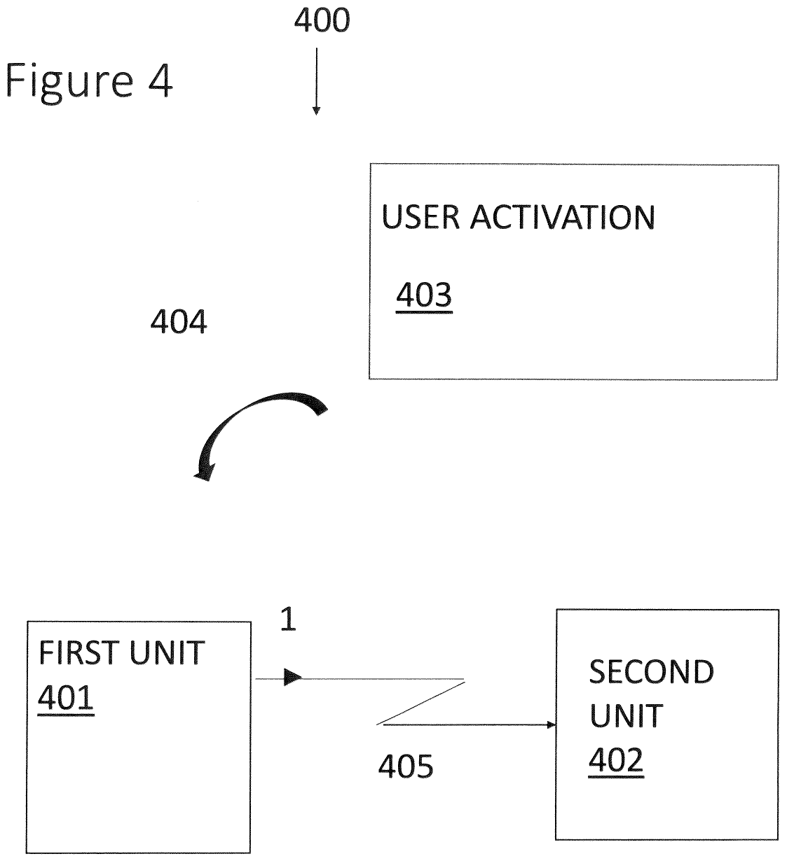

FIG. 4 is a block diagram of a pair of whispers including the first and second units which are wirelessly connected to each other, and user activation in which the communication protocol between the first and the second whisper includes one “handshake” initiated by the first whisper that includes information on both duration and intensity of the pulse;

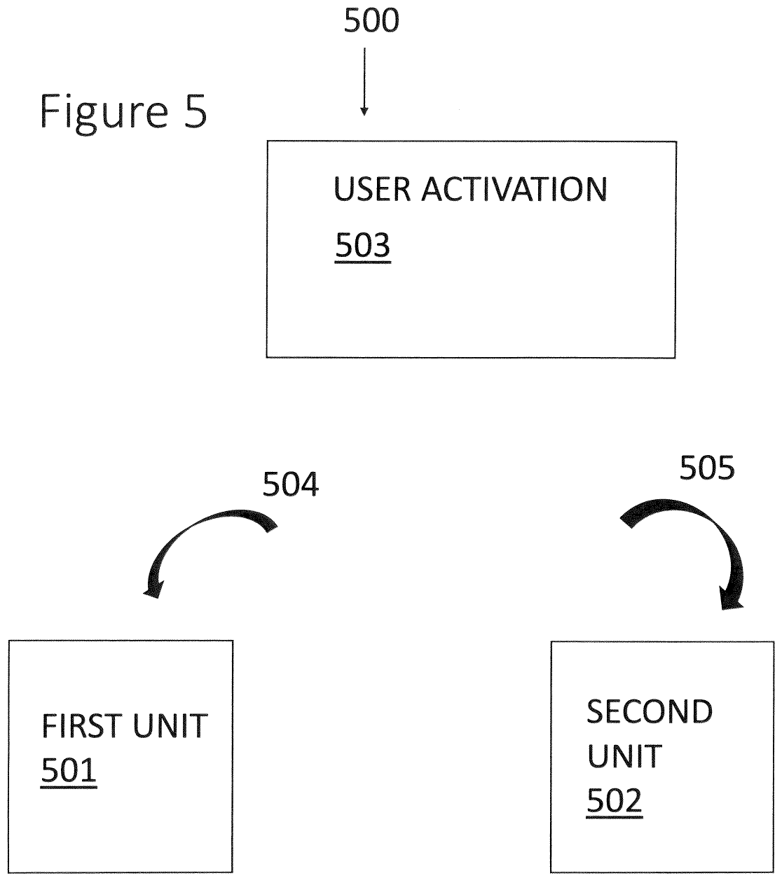

FIG. 5 is a block diagram of a pair of whispers including the first and second units which are wirelessly connected to each other, and user activation in which the user sets the duration and intensity of the pulse of each whisper, respectively;

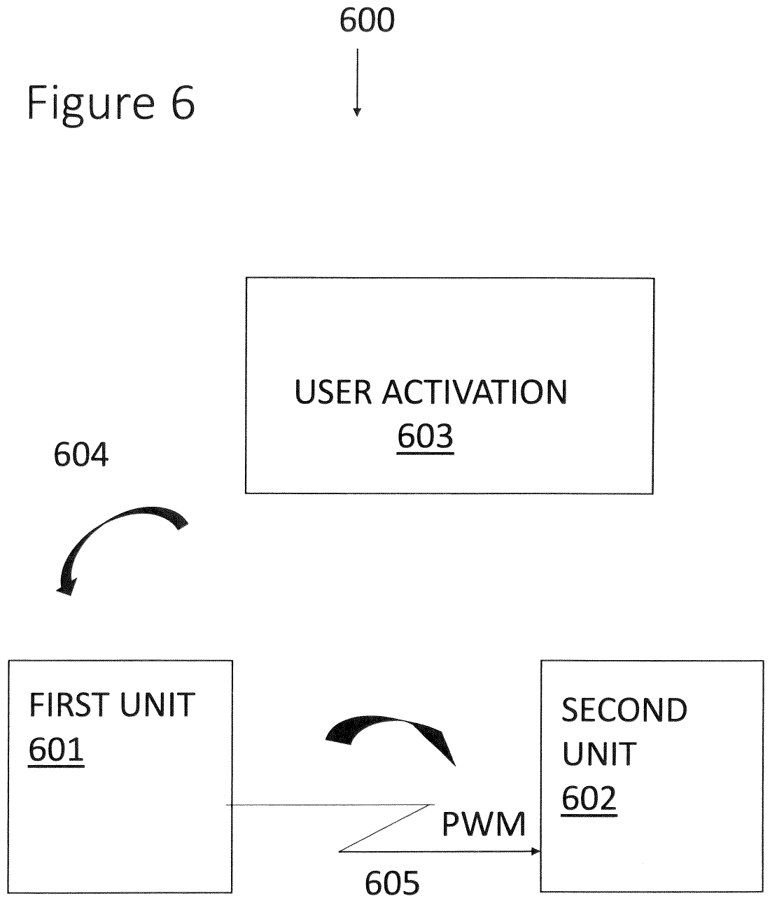

FIG. 6 is a block diagram of a pair of whispers including the first and second units which are wirelessly connected to each other, and user activation in which the communication protocol between the first and the second whisper includes information on duration and intensity of the pulse, respectively, encoded within a pulse width modulation signal;

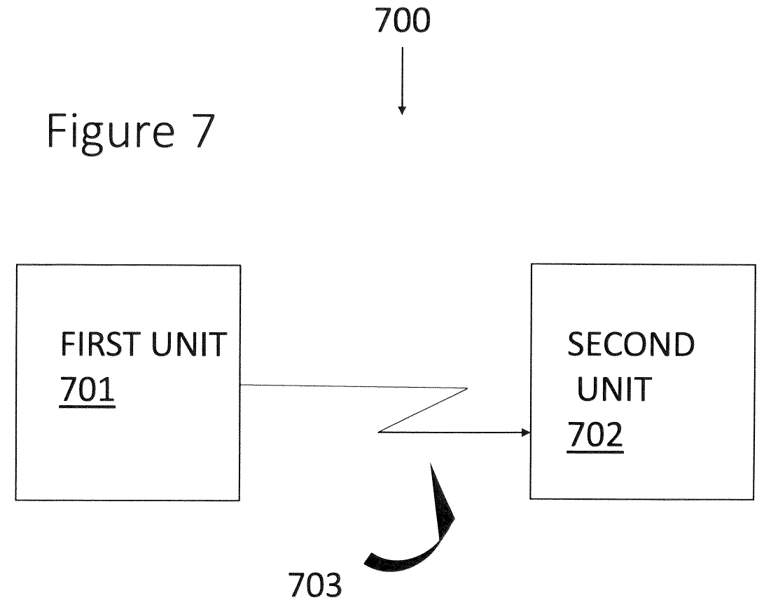

FIG. 7 is a block diagram of a first unit which is transmitting a wireless signal to a second unit to initiate pairing and synchronization;

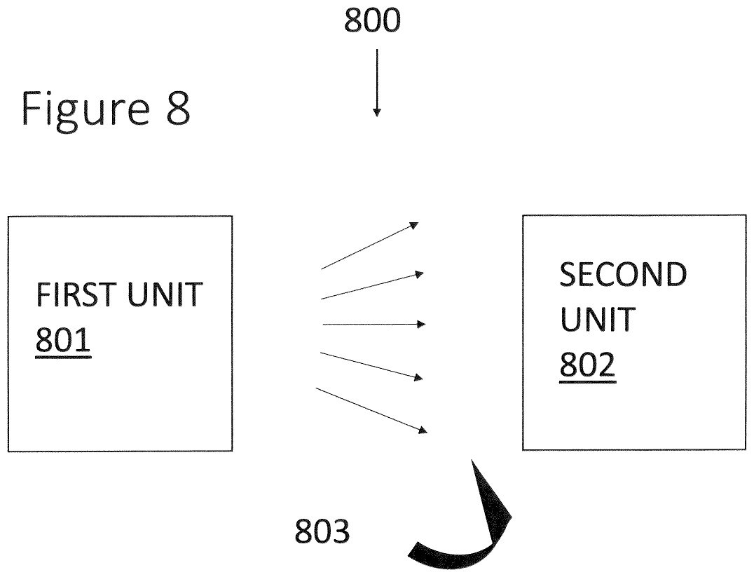

FIG. 8 is a block diagram of a first unit which is transmitting an infrared optical signal to a second unit to initiate pairing and synchronization;

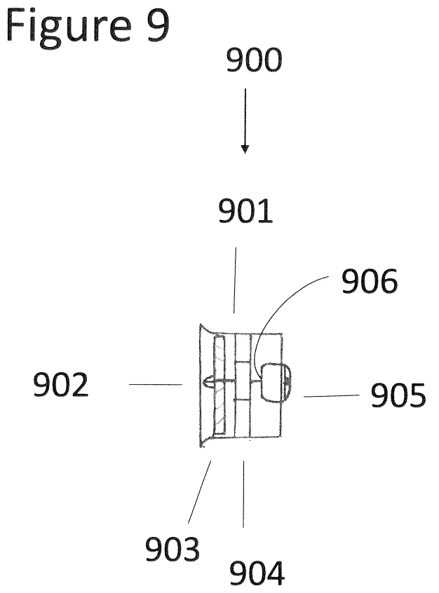

FIG. 9 is an isometric view of an EDF with a standard duct profile;

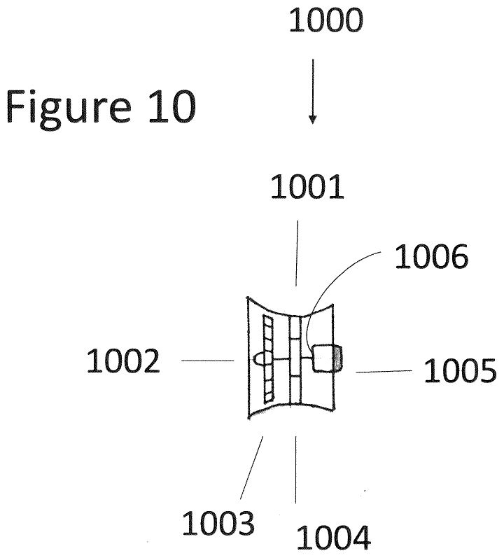

FIG. 10 is an isometric view of an EDF with a Venturi duct profile;

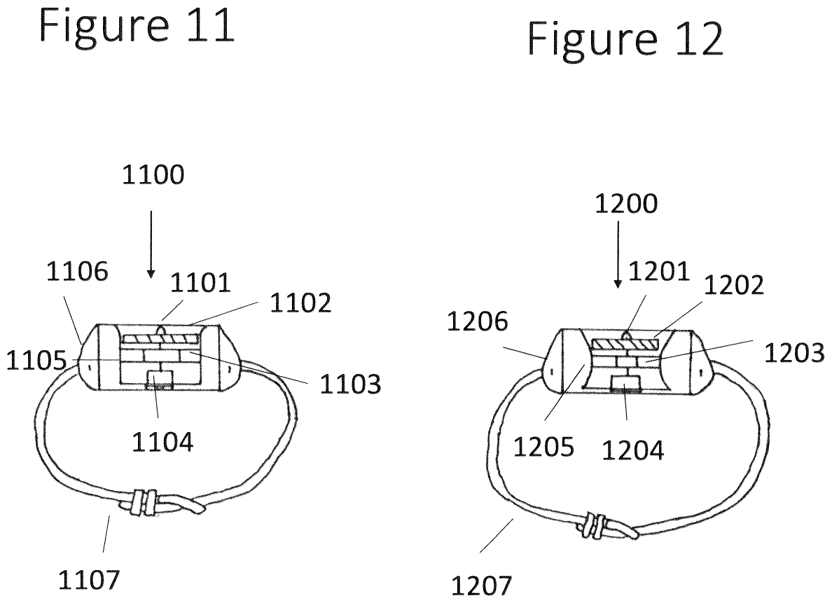

FIG. 11 is a schematic diagram of a whisper utilizing an EDF with a standard duct profile, whereby the EDF generates a pressure differential to induce bilateral indirect cutaneous stimulation;

FIG. 12 is a schematic diagram of a whisper utilizing an EDF with a venturi duct profile, whereby the EDF generates a pressure differential to induce bilateral indirect cutaneous stimulation;

FIG. 13 is a schematic diagram of a window and nonluminous roller to produce bilateral nonluminous visual stimulation;

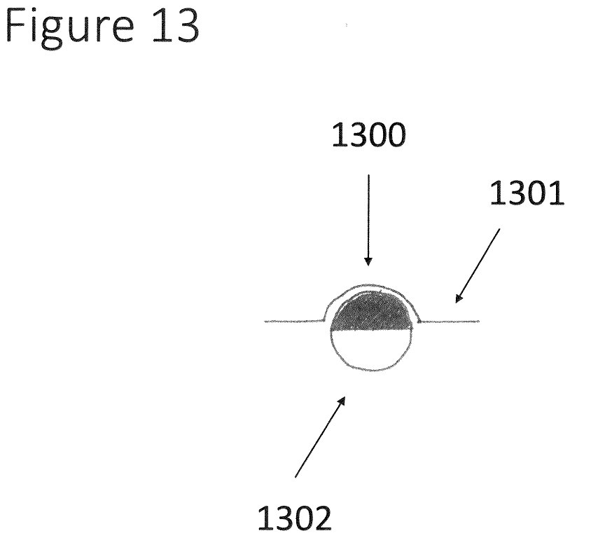

FIG. 14 is an isometric view of a window and nonluminous roller to produce bilateral nonluminous visual stimulation;

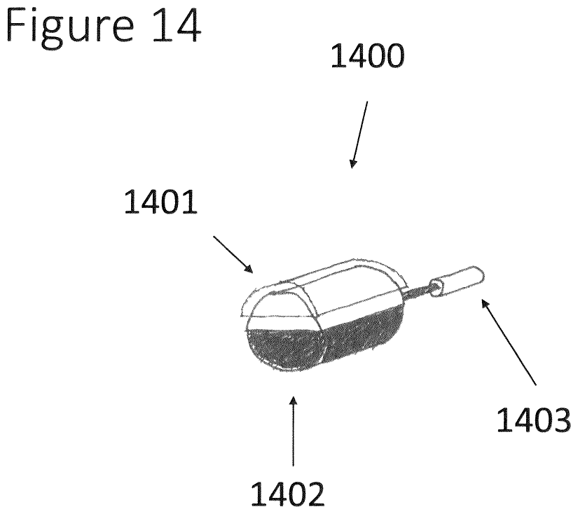

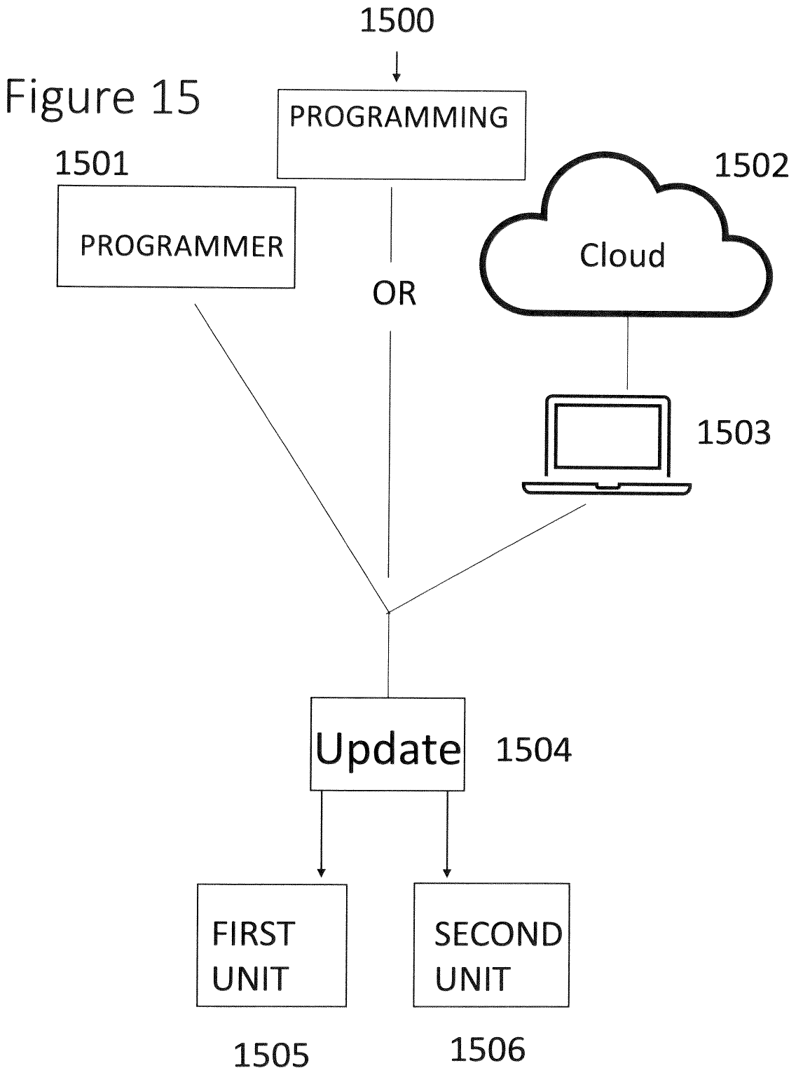

FIG. 15 is a block diagram showing the updating of firmware of a pair of whispers via: 1) a wired connection to a personal computer using firmware updates from the cloud; and 2) an industrial chip programmer;

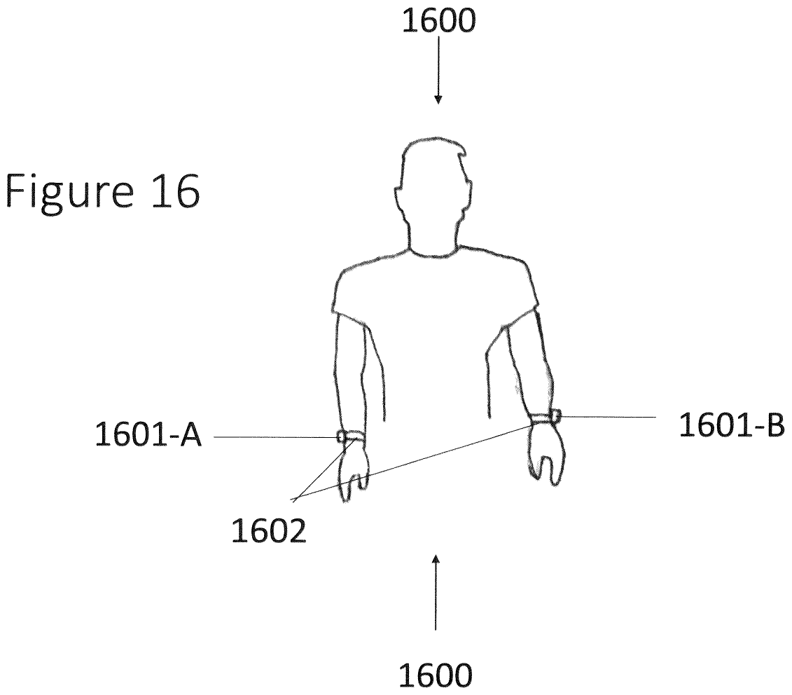

FIG. 16 is a diagram of a pair of whispers worn on each wrist by a subject to provide bilateral indirect cutaneous stimulation;

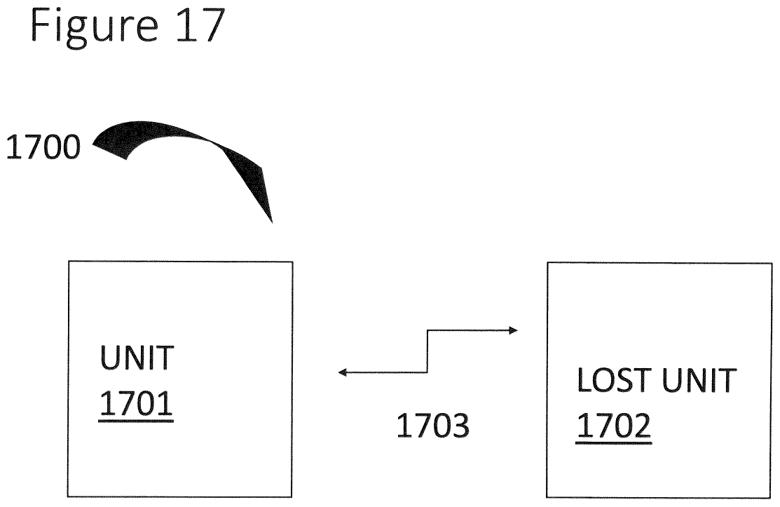

FIG. 17 is a block diagram that depicts a protocol for finding a lost whisper;

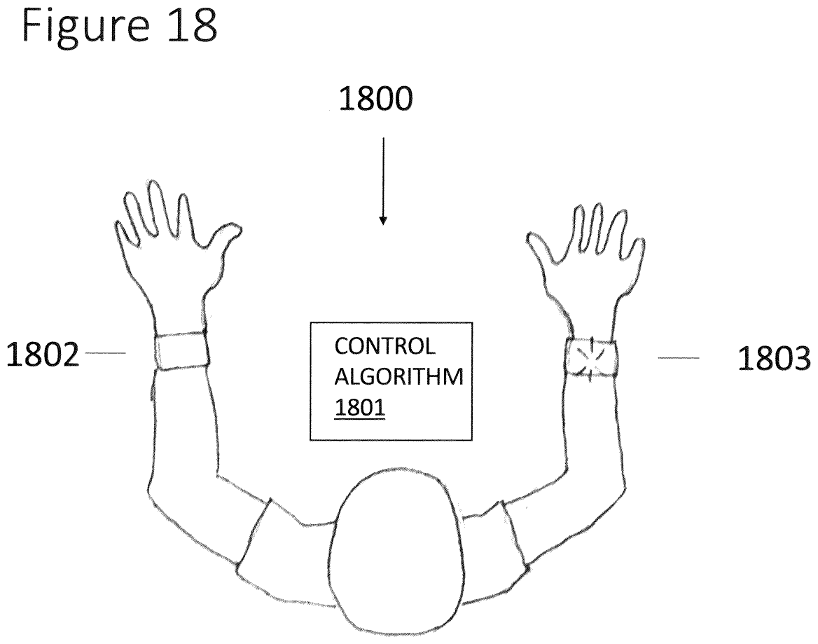

FIG. 18 is a block diagram which bilateral nonluminous visual stimulation through a pair of whispers;

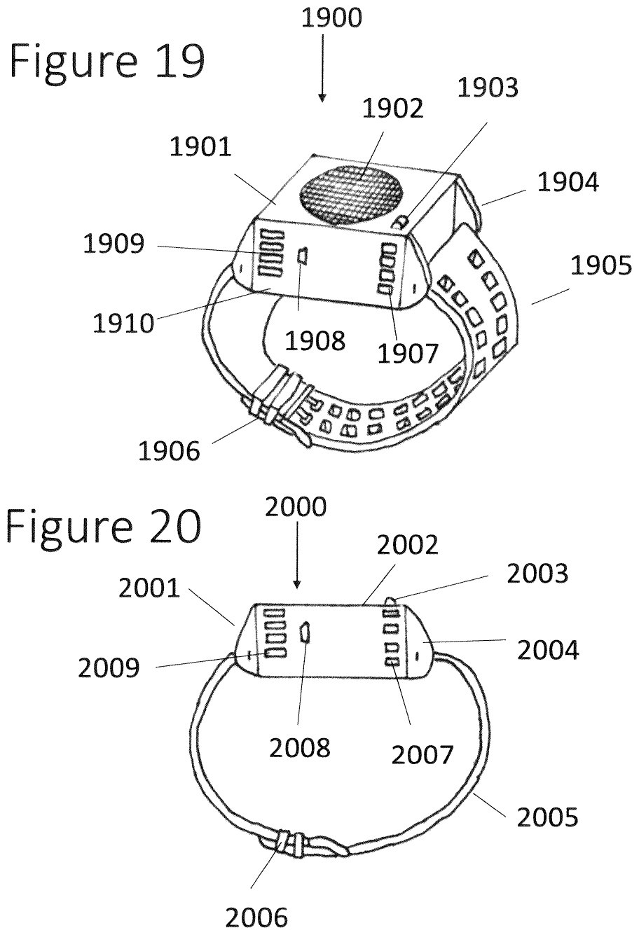

FIG. 19 is an isometric view from an elevated vantage point of a whisper;

FIG. 20 is a front view of the whisper of FIG. 19;

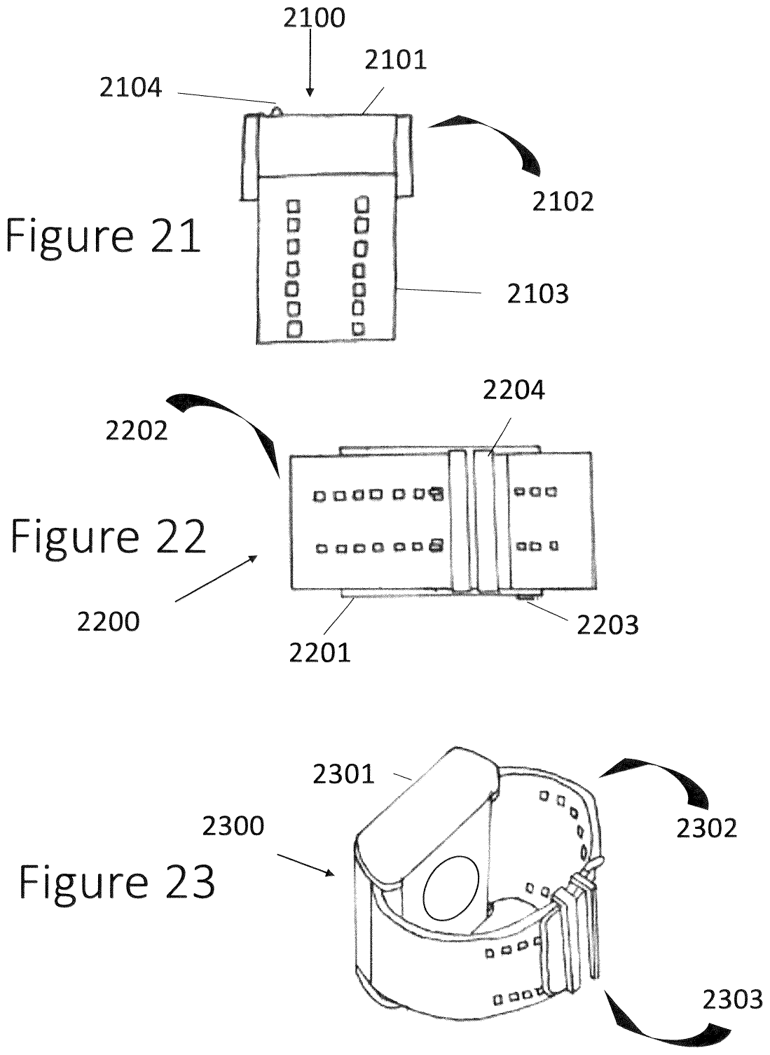

FIG. 21 is a left-side view of the whisper of FIG. 19;

FIG. 22 is a bottom view of the whisper of FIG. 19;

FIG. 23 is an isometric view from a lower/oblique vantage point of the whisper of FIG. 19;

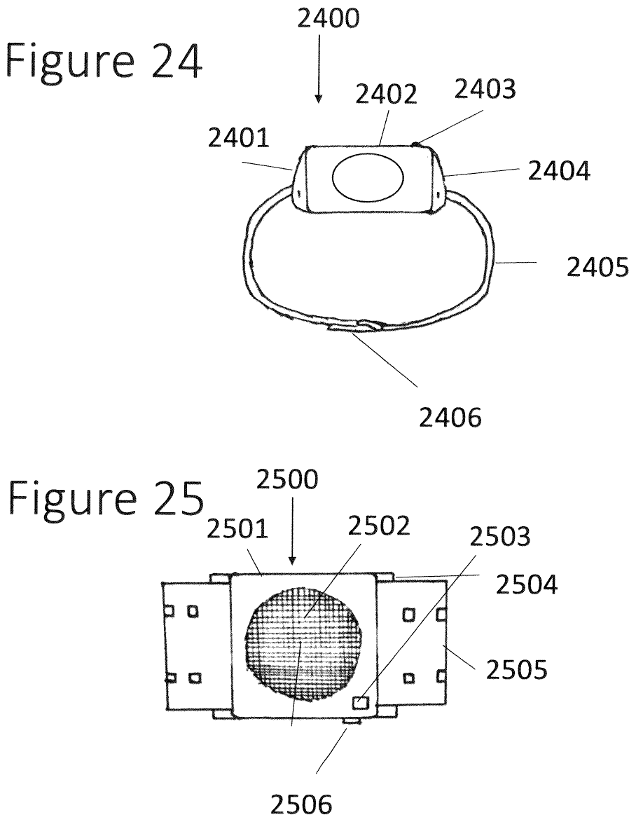

FIG. 24 is a front view of the whisper of FIG. 19;

FIG. 25 is top view of the whisper of FIG. 19;

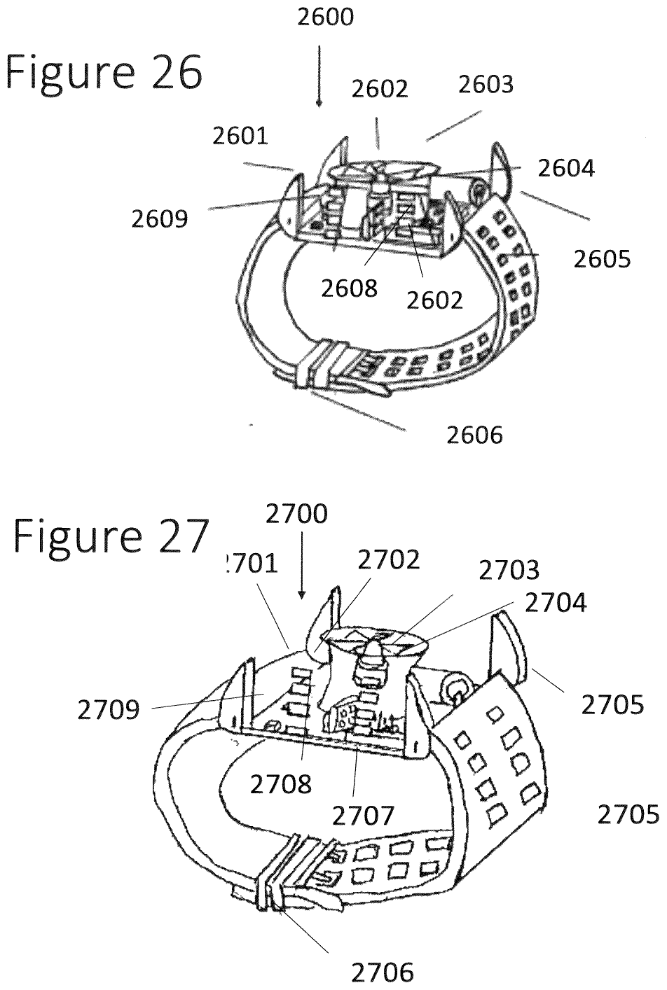

FIG. 26 is an isometric view of the whisper of FIG. 19, with the cover removed to show the internal components, which include an EDF with a standard duct profile, taken from an elevated/oblique vantage point;

FIG. 27 is an isometric view of the whisper of FIG. 19, with the cover removed to show the internal components, which include an EDF with a venturi duct profile, taken from an elevated/oblique vantage point;

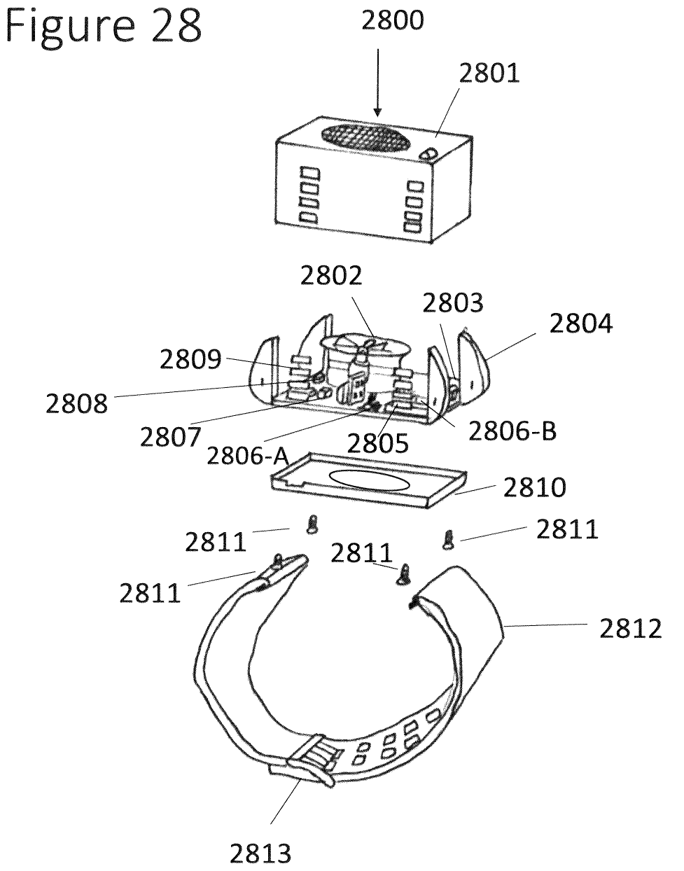

FIG. 28 is an isometric exploded view of the whisper of FIG. 26, taken from an elevated/oblique vantage point;

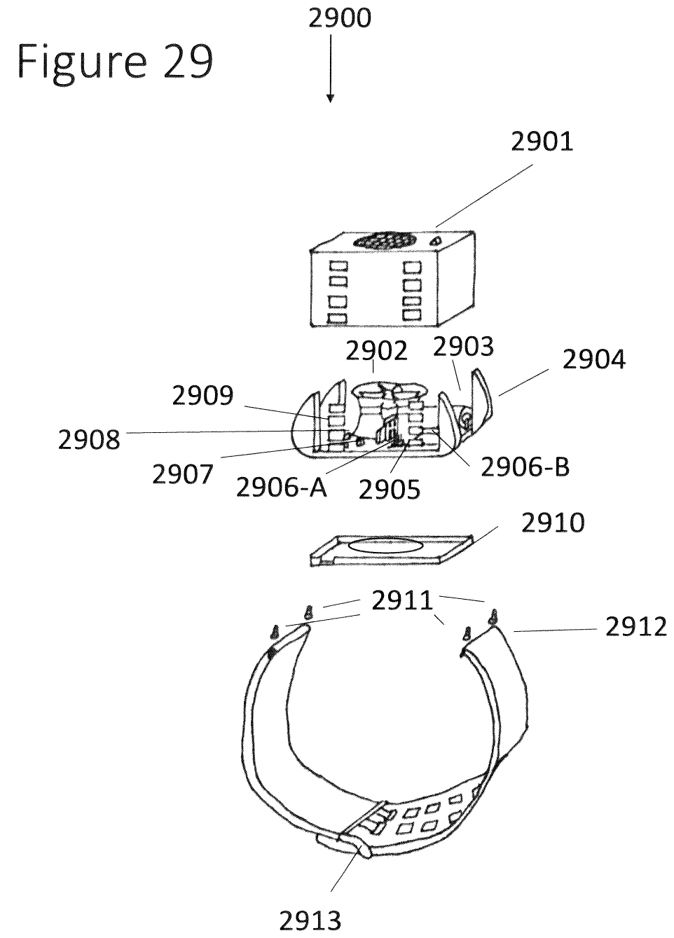

FIG. 29 is an isometric exploded view of the whisper of FIG. 27, taken from an elevated/oblique vantage point; and

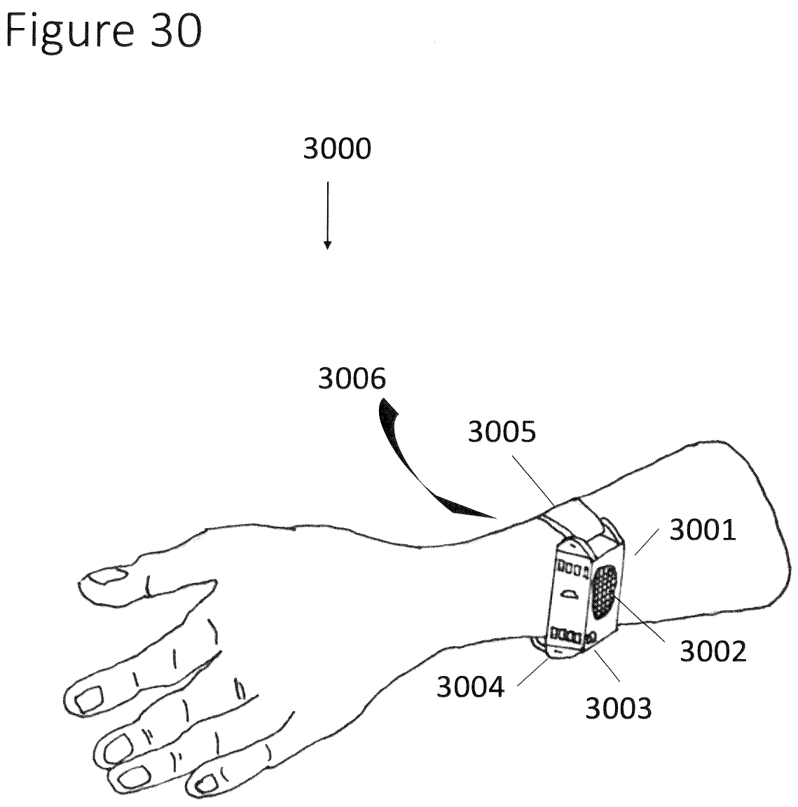

FIG. 30 is an isometric view of a whisper as worn on the left arm of a person.

### Feature Description

### DETAILED DESCRIPTION OF THE INVENTION

Referring now to FIG. 1, a preferred embodiment of an indirect cutaneous stimulation unit (also referred to herein as a whisper) is shown.

Referring now to FIG. 2, 200 of the apparatus for administering bilateral indirect cutaneous stimulation to a human subject comprises a wireless transceiver 201, a microcontroller 202, an electric ducted fan (“EDF”) 203, an electronic speed controller (“ESC”) 204 for driving the EDF 203 and optionally roller 213, a battery 205, a charging/protection circuit 206, which controls charging of the battery and protects against over-discharge, over-charge, and over-voltage, a power supply 207, a user-accessible external connector 208, a control element which, in this case is a normally open momentary contact switch (N.O. MCS) 209, an LED bar status indicator 210, an optional programming connector 211, and an antenna 212, all of which reside in a housing 214. A whisper can be worn on the wrist by means of a strap, or placed in pockets. The EDF 203 is controlled exclusively by the ESC 204. The EDF 203 converts electrical energy, provided by the battery 205, into air flow. When the EDF 203 is energized by the ESC 204, electrical power is delivered, over time, to EDF 203, and that power is used to create the air flow which is perceived as a cutaneous touch and/or pressure by the human subject.

Typically, the battery 205 is a secondary-type cell, which allows it to be recharged after it has become depleted. A presently preferred battery 205 uses Lithium-Polymer (LiPo.sub.4) chemistry. Other chemistries can be used, but the LiPo is presently considered to be optimum in terms of power-density, cost and safety.

It is important to recharge the on-board battery through the charging/protection circuit 206. The charging/protection block 206 optimizes charging for the chemistry of the battery 205. It controls the voltage and amperage of the charge both maximize the charge speed, preserve longevity of the battery, and prevent dangers associated with overcharging of the battery 205.

The power supply 207 regulates and smooths the voltage to the other blocks in the diagram of FIG. 2. Different blocks require different voltages. Thus, the primary function of the power supply 207 is to provide the appropriate regulated voltage to these blocks.

The external connector 208 enables a user to charge the battery 205 with a USB cable from a 5V power source. The connector 208 can also be used to provide software updates to the microcontroller 202. Substantially all of the connector is inside the housing 213, with only a port to access the connector exposed. The exposed port allows sufficient accesses to the connector to connect an external charging/communication cable.

The wireless transceiver 201 facilitates wireless communication with other whispers. The wireless transceiver 201 communicates with the microcontroller 202 digitally through an inter-integrated (I2C), universal asynchronous receive/transmit (UART), or serial peripheral interface (SPI) protocol. The preferred embodiment for the wireless transceiver 201 avoids standard communication protocols found in the vast majority of devices connected to the internet of things (IOT) such as Bluetooth and Wi-Fi.

The whispers produce bilateral cutaneous stimulation through airflow that is perceived as a cutaneous touch or pressure. As the whisper is activated, the EDF is rotated as electrical energy is transformed into mechanical energy. As EDF undergoes circular acceleration, a force is exerted on the air within the duct, according to Newton and Bernoulli's laws. As a result, the air in the duct is accelerated toward the wrist of the user, gaining kinetic energy as it is accelerated. This energy is ultimately conducted to the skin of the user and the nerve endings thereunder. The profile of the airflow (speed, duration, pressure, etc.) are adjusted to optimize the bilateral indirect cutaneous stimulation of the user.

The EDF 203 converts electrical energy into rotational energy. When it is energized by the ESC 204, electrical power is delivered, over time, to the EDF 203, and that power is used to create airflow.

The ESC 204 primarily consumes energy from the power supply 207 with only a negligible amount energy coming from the microcontroller 202. Energy delivered by the microcontroller 202 is for the purpose of communicating with the ESC 204. For more intense sensations, the ESC 204 cycles the EDF 103 at higher Revolutions Per Minute (RPM), thus consuming a relatively large amount of energy. As a result, users experience more intense cutaneous stimulation. For more gentle cutaneous stimulation, the ESC 204 cycles the EDF 203 at a lower RPM, thus consuming a relatively low amount of energy.

1. For the most intense cutaneous stimulation, the EDF 203 is driven by the ESC 204 at the maximum RPM as set within the bounds of performance programmed into the microcontroller (202).

2. For moderate cutaneous stimulation, the EDF 203 is driven by the ESC 204 at a moderate RPM. The RPM of the EDF 203 can be adjusted through the timing of the ESC channel cycling.

3. For the least intense cutaneous stimulation, the EDF 203 is driven by the ESC 204 at the minimum RPM as set within the bounds of performance programmed into the microcontroller (202).

a. An additional method may be utilized to augment the intensity of cutaneous stimulation. The acceleration of the air column in the EDF 203 may be sharp or gradual depending on how rapidly the ECS 204 accelerates the EDF 203. Stated otherwise, the air column in the EDF 203 may be accelerated gradually or sharply. A more rapid acceleration of the air column will be experienced by the user as a more intense cutaneous stimulation.

In all states, the operation of the ESC 204 is controlled by the microcontroller 202. The microcontroller 202 is powered by the power supply 207. The microcontroller 202 directs operation of the entire whisper. Control of the EDF 203 is provided by a pulse width modulation (PWM) signal 202, timed by a crystal oscillator, to the ECS 204 which translates the PWM signal to electrical energy acting on the stator coils of the EDF 203. When the whisper has been turned off, the microcontroller 202 conserves energy by entering a “sleep” mode with lower energy consumption from the battery 205. While in sleep mode, the battery 205 will better conserve charge. When turned on by depression of the momentary contact switch (MCS) 209 the whisper's microcontroller 202 exits sleep mode as indicated visually by LED indicator 210. The whisper also provides feedback through the EDF 203. The microcontroller 202 is programmed through the optional programming connector 211 or the external connector 208.

Additional aspects of the invention are shown in FIG. 3. A user activation 303 turns a whisper (first unit 304) on. The first unit 304 will then, communicate wirelessly with a second whisper (second unit 302), which synchronizes itself with the first unit 304 via wireless signal with two handshakes. Indirect cutaneous stimulation is achieved by alternating the first unit 304 on one side of the user, with the second unit 302 on the other side, cycling back and forth between the whispers 304 and 302. The preferred speed of the cycle varies from one user to another as does the intensity. While some users desire intense stimulation, others require minimal stimulation. The timing of the back and forth bilateral stimulation cycles influences the effectiveness of the therapy. The effectiveness of the therapy depends on optimizing the rate for rate for each user, and may vary for individual users depending on the conditions they experience. The intensity of the cutaneous stimulation also impacts the effectiveness of the therapy. Certain users need relatively intense stimulation to benefit from the therapy, others require relatively mild stimulation. The user activation 303 can be used to set the rate of acceleration of the air flow, duration of air flow, and rate of cycle of cutaneous stimulation. The user activation does so by cycling the MCS through preselected settings.

Referring now to FIG. 4, an alternate embodiment of the whisper system is shown where a single handshake in the wireless signal 405, from the first unit 401, designated by the user activation 403 interfaces and synchronizes with the second unit 402.

Referring now to FIG. 5, a second alternate embodiment of the whisper system is shown where a the first unit 501 is synchronized with the second unit 502 without a wireless signal, using user activation 503 instead.

Referring now to FIG. 6, a third alternate embodiment of the whisper system is shown where a PWM wireless signal 605, from the first unit 601, designated by the user activation 603 interfaces and synchronizes with the second unit 602.

Referring now to FIG. 7, after the first unit 701 is, turned on, it “listens” for another unit. If it does not detect another unit transmitting, it assumes the role of first unit and transmits to the second unit 702 with a wireless signal 703 to synchronize the bilateral cutaneous stimulation cycle. The first unit 701 and the second unit 702 remain maintain synchronization by the wireless signal 703 from the first unit.

Referring now to FIG. 8, an alternate embodiment of FIG. 7 depicts a first unit 801 and a second unit 802, in which an infrared signal 803 is used to synchronize the two units.

Referring now to FIG. 9, a preferred embodiment 203-A of the EDF 203 of FIG. 2 consists of a duct, fan mounted on a driveshaft within the duct (standard profile), and a brushless electric motor (composed of stator coils and a polarized magnetic rotor which divides rotation into a number of discrete steps) mounted to the duct to turn the drive shaft. Together these components of the EDF accelerate the column of air within the duct. As the column of air accelerates through the gap between the user's skin and the exit of the duct, dynamic pressure increases, resulting in a loss of static pressure experienced by the user as the sensation of touch. In an alternative embodiment, as the column of air decelerates when it contacts the user's skin, static pressure increases and is experienced by the user as the sensation of pressure. The pressure experienced by the user is static pressure, which is inversely related to dynamic pressure, which is the cause of the acceleration of the column of air imparted by the EDF. As the EDF turns, the blades create lift which imparts dynamic pressure to the column of air within the duct. As the air moves through the duct, it ultimately accelerates between the user's skin and the exit of the duct, decelerates and stops when it meets the user's skin. In the case of accelerator, the state pressure decreases as the dynamic pressure increases. Or, in the alternative embodiment, as a result of the deceleration, dynamic pressure decreases, and static pressure increases. This is a function of the Law of Continuity. Conceptually this is the equivalent to feeling the blast of a fan on one's face. The relationship between dynamic and static pressure is described thus: ½ qV.sup.2=C−p, where q is density, V is velocity, C is a constant, and p is static pressure. Static pressure is typically measured in the SI unit of pascals. When the EDF 203-A of FIG. 9 is used in a whisper 200, the application of pressure can be generated by rotationally accelerating the fan 803, and consequently the column of air within the duct 901. When power is applied to the stator coils of the stepper motor 906, mounted on a driveshaft 902, which in turn, is mounted to supports 904 connected to the duct 901, the energized stator coils 906 on the exterior of the stepper motor create an electromagnetic force that acts on the magnetic rotor 905 and turn the fan 903. The blades of the fan 903 create lift and accelerate the column of air within the duct 901. As the whisper 200 is pressed against the users skin, the user's skin acts as a barrier impeding and thus in the first embodiment, accelerating the column of air between the user's skin and the duct exit, or in the second embodiment, decelerating the column of air within the duct 901. The acceleration rate, speed, and duration of the activation of EDF 203 by the ECS 204 allows for tailoring of the cutaneous stimulation received by the user. The majority of users prefer less intense stimulation from the whisper 200. Less intense cutaneous stimulation can be elicited through lower RPM operation of the stepper motor 906 and fan 903, and gradual acceleration/deceleration of same. Those who require or prefer more intense cutaneous stimulation can achieve same through the operation of the stepper motor 906 and fan at higher RPMs and a higher rate of acceleration/deceleration. The microcontroller 202 includes programming that allows the ECS 204 to operate with customizable parameters, such as the rate of acceleration, RPM, and duration of operation of the EDF 203. The profile of such parameters operation can be programed as a function (i.e., linear), or nonfunctional. This allows for the EDF 203 to specifically tailor the cutaneous stimulation to the user. Alternatively, the terminus of the duct may be placed close enough to the user's skin that the column of air is forced through a narrow gap between the duct and the user's skin at a high velocity, thus reducing static pressure with the corresponding increase in dynamic pressure, resulting in cutaneous stimulation through a negative pressure differential.

Referring now to FIG. 10, a preferred embodiment 203-B of the EDF 203 of FIG. 2 within the consists of a duct, fan mounted on a driveshaft within the duct (Venturi profile), and a brushless electric motor (composed of stator coils and a polarized magnetic rotor which divides rotation into a number of discrete steps) mounted to the duct to turn the drive shaft. The together these components of the EDF accelerate the column of air duct. As the column of air accelerates through the gap between the user's skin and the exit of the duct, dynamic pressure increases, resulting in a loss of static pressure experienced by the user as the sensation of touch. In an alternative embodiment, as the column of air decelerates when it contacts the user's skin, static pressure increases and is experienced by the user as the sensation of pressure. The pressure experienced by the user is static pressure, which is inversely related to dynamic pressure, which is the cause of the acceleration of the column of air imparted by the EDF. As the EDF turns, the blades create lift which imparts dynamic pressure to the column of air within the duct. As the air moves through the duct, it ultimately accelerates between the user's skin and the exit of the duct., decelerates and stops when it meets the user's skin. In the case of acceleration, the state pressure decreases as the dynamic pressure increases. Or, in the alternative embodiment, as a result of the deceleration, dynamic pressure decreases, and static pressure increases. This is a function of the Law of Continuity. Conceptually this is the equivalent to feeling the blast of a fan on one's face. The relationship between dynamic and static pressure is described thus: ½ qV.sup.2=C−p, where q is density, V is velocity, C is a constant, and p is static pressure. Static pressure is typically measured in the SI unit of pascals. When the EDF 203-B of FIG. 2 is used in a whisper 200, the application of pressure can be generated by rotationally accelerating the fan 1003, and consequently the column of air within the duct 1001. When power is applied to the stator coils of the stepper motor 1006, mounted on a driveshaft 1002, which in turn, is mounted to supports 1004 connected to the duct 1001, the energized stator coils 1006 on the exterior of the stepper motor create an electromagnetic force that acts on the magnetic rotor 1005 and turn the fan 1003. The blades of the fan 1003 create lift and accelerate the column of air within the duct 1001. As the whisper 200 is pressed against the users skin, the user's skin acts as a barrier impeding and thus in the first embodiment accelerating the column of air between the user's skin and the duct exit, or in the second embodiment, decelerating the column of air within the duct 1001. The acceleration rate, speed, and duration of the activation of EDF 203 by the ECS 204 allows for tailoring of the cutaneous stimulation received by the user. The majority of users prefer less intense stimulation from the whisper 200. Less intense cutaneous stimulation can be elicited through lower RPM operation of the stepper motor 1006 and fan 1003, and gradual acceleration/deceleration of same. Those who require or prefer more intense cutaneous stimulation can achieve same through the operation of the stepper motor 1006 and fan at higher RPMs and a higher rate of acceleration/deceleration. The microcontroller 202 includes programming that allows the ECS 204 to operate with customizable parameters, such as the rate of acceleration, RPM, and duration of operation of the EDF 203. The profile of such parameters operation can be programed as a function (i.e., linear), or nonfunctional. This allows for the EDF 203 to specifically tailor the cutaneous stimulation to the user.

Still referring to FIG. 10, embodiment 203-B differs from embodiment 203-A in that embodiment 203-B has the duct with a Venturi profile while embodiment 203-A has a duct with a standard profile.

Referring now to FIG. 11, a schematic diagram of a whisper employing embodiment 203-A is shown.

Referring now to FIG. 12, a schematic diagram of a whisper employing embodiment 203-B is shown.

Referring now to FIG. 13, a schematic diagram for the nonluminous roller and window providing nonluminous roller 213 to produce bilateral nonluminous visual stimulation for a second embodiment of whisper 200. A window and nonluminous roller 213 is displayed on the whisper 200. The roller is turned by a stepper motor in increments of 180 degrees, so that the user sees either an all white or all black display through the window. The roller display is synchronized with the EDF tactile stimulation, for the purpose of allowing the user to experience nonluminous visual stimulation in which the eyes of the user pan back and forth between the first unit and second unit. The EDF may be turned off so that the user may experience nonluminous visual stimulation only.

Referring now to FIG. 14, Roller 1400 consists of a bisected cylinder 1402 mounted under a transparent window 1401, with nonluminous white coloration on one half, and flat black coloration on the other half, mounted to a driveshaft, and a brushless electric motor 1403 (composed of stator coils and a polarized magnetic rotor which divides rotation into a number of discrete steps) mounted to the duct to turn the drive shaft. When power is applied to the stator coils of the stepper motor 1403, the energized stator coils on the exterior of the stepper motor create an electromagnetic force that acts on the magnetic rotor and turns the drum. They energization of the stator coils is controlled by microcontroller 202 in such a way as to rotate the cylinder 1402 180 degrees, displaying either all white, or all black in window 1401. The alternating 180 rotations of cylinder 1402 in two units, when tracked by the eyes of a user, creates bilateral nonluminous visual stimulation.

Referring now to FIG. 15, provision has been made for two methods of updating the software running in the microcontroller 202 running a first unit and a second unit respectively. To update the software, a USB cable is used to attach the optional programming connector 211 (which may use the same port as the external charging connector 208) to microcontroller 202. An industrial programmer 1501, or alternatively a computer 1503 obtaining the update 1504 from the cloud 1502, can be used to install the update 1504 in the user's first unit 1505 and second unit 1506 respectively.

Still referring to FIG. 15, the update 1504 is preloaded onto the industrial programmer 1501, or uploaded to the cloud 1502 where it can be accessed and downloaded by a computer 1503.

To experience bilateral indirect cutaneous stimulation, whispers must be attached to a user's body with the included bands. A user need not use the bands for bilateral nonluminous visual stimulation, but can instead hold the whispers or set them down. This embodiment will be shown extensively in the attached drawing FIGS. 19 through 30.

Referring now to FIG. 16, a pair of whispers 1601-A and 1601-B are each attached to a band 1602, which can be worn about the wrist, arm, or ankle so that a user can experience bilateral indirect cutaneous stimulation.

Referring now to FIG. 17, it is contemplated that an individual may inadvertently lose or misplace one whisper. In such an event, the user can use the remaining unit 1701 to communicate with the lost unit 1702 by way of a wireless signal 1703 to activate the lost unit 1702. The remaining unit 1701 gives the user feedback as they get closer to or further from the lost whisper 1702 by emitting a tone through the EDF 203. The EDFs such as EDF 203 are commonly used to make tones. The mechanism for making such a tone requires a signal from the microcontroller 202, and sent to the ESC 204, which is calibrated to turn the magnetic rotor 1005 back and forth a small amount by energizing the stator coils 1006 to pull the magnetic rotor 1005 first one way, and then the other. The frequency used to rotate the magnetic rotor 1005 back and forth is a frequency within the spectrum of frequencies that can be detected by ear. Thus the mechanism uses the ECS 204 to drive the stator coils 1006 which act on the magnetic rotor 1004 which then functions as a speaker.

Referring now to FIG. 18, an embodiment of the bilateral nonluminous visual stimulation is depicted. In this embodiment, the user 1800 is depicted wearing a whisper 1802, 1803, on each wrist. The control algorithm 1801 of the self-synchronized whispers, first unit 1802 and second unit 1803, rotates bisected cylinder 1402 on first unit 1802 so that the dark side is face up and visible through the window 1401 on the first unit 1802, while simultaneously rotating bisected cylinder 1402 on second unit 1803 so that the bright side is face up and visible through the window 1401 on the second unit 1803. The control algorithm 1801 then alternates the arrangement so that the first unit 1802 displays the bright side of bisected cylinder 1402 through the window 1401 on the first unit 1802, while the second unit 1803 displays the dark side of bisected cylinder 1402 through the window 1401 on the second unit 1803. The user pans his or her eyes back and forth, focusing them on whichever unit displays the bright side of that units bisected cylinder 1402 in its window 1401, thus producing bilateral nonluminous visual stimulation.

Referring now to FIGS. 19 through 25, a presently preferred embodiment of a whisper 1900 is configured with a wrist strap 1905 that is secured by a buckle 1906. The whisper 1900 utilizes a connector 1908 for charging and communication. A status indicator 1910, which displays status indicator LED 210, gives the user visual feedback as to the operational status of the whisper 1900. An array of four buttons 1907 enables the user to turn the whisper on and off, and control the settings of whisper 1900. The whisper body has a top cover 1901, which fits over a chassis 1910.

Referring now to FIG. 26, a preferred embodiment whisper 2600 is shown with its top cover 1901 removed, thereby exposing the internal components of the whisper. The EDF 2602, status indicator 1909 (in FIG. 26 labeled as 2609) and button array 1907 (in FIG. 26 labeled as 2607) are visible in this view. The top cover 1901 has been removed to expose the interior components. The EDF 203-A is visible, which in this case incorporates a standard profile duct 901. A battery 205 powers the whisper 2600. The battery 205 in the preferred embodiment is a lithium polymer (LiPo) battery. Other, more advanced battery chemistries may be contemplated. The LiPo is preferred because it offers better energy density than Pb, NiCd and NiMH chemistries, while remaining relatively inexpensive. LiPo presents a low risk of fire with proper charge maintenance. The micro-controller 202 is also shown.

Referring now to FIG. 27, an alternative embodiment whisper 2700 is shown with its top cover 1901 removed, thereby exposing the internal components of the whisper. This alternative embodiment whisper 2700 is similar to embodiment 2600, with the exception that the EDF 203-B is incorporates a Venturi duct profile.

Referring now to FIG. 28, the tapper 2600 of FIG. 26 is shown in an exploded view. The EDF 203-A is secured to the circuit board 2807. The circuit board assembly 2804, with all components soldered to the circuit board 2807, has been removed from the base 2810. Thus, a preferred embodiment whisper includes a base 2810, a top cover 2801, a circuit board assembly 2804 incorporating an EDF 203-A, an array of four user pressable buttons 2805 that activates the normally-open momentary contact switch 209, and a status indicator 1909 (in FIG. 28 labeled as 2809) that displays the status indicator LED 210. The entire whisper is held together with four machine screws 2811. The wristband 2812 has a buckle 2813.

Referring now to FIG. 29, the whisper 2700 of FIG. 27 is shown in an exploded view. This embodiment whisper 2700 is similar to the whisper 2600 and 2800 of FIGS. 26 and 28, respectively, with the primary difference being substitution of EDF 203-B with a Venturi profile duct instead of the standard profile duct of EDF 203-A.

Referring now to FIG. 30, a whisper 1900 is shown secured to a user's arm 3000 with a wristband 1905 (in FIG. 30 labeled as 3005).

Although only preferred embodiments of the apparatus for administering bilateral indirect cutaneous stimulation have been depicted and described, herein, alterations and modifications may be made due to engineering necessities without departing from the purview, nature, and functionality of the invention as hereinafter claimed.

1. An apparatus for administering bilateral indirect cutaneous stimulation to a human subject, said device comprising: a pair of self-synchronizing units to apply stimulation events; a first cutaneous stimulation unit powered by a first rechargeable battery in contact with one side of the human subject, said first unit containing a first micro-controller, a first electric ducted fan (“EDF”), and a first transceiver that communicates with the second unit; a second cutaneous stimulation unit powered by a second rechargeable battery in contact with the other side of the human subject, said second unit containing a second micro-controller, a second EDF, and a second transceiver that communicates with the first unit; and wherein each said EDF is an electric ducted fan, consisting of a duct, fan mounted on a driveshaft within the duct, and a brushless electric motor (composed of stator coils and a polarized magnetic rotor which divides rotation into a number of discrete steps) mounted to the duct to turn the drive shaft; and wherein the stator coils are fed a pulsed flow of electricity, each stator coil generates a magnetic field that applies a torque to the rotor, said torque producing an opposite and equal reverse torque that turns the fan of the unit of which the motor is a part, forcing a flow of air through the duct against the skin of the person wearing such unit, said airflow being perceived as a cutaneous touch and/or pressure differential by the human subject.

2. The apparatus for administering bilateral indirect cutaneous stimulation subject of claim 1, wherein the EDF of each cutaneous stimulation unit is attached to a brushless “stepper” motor in which the rotation is divided into discrete controllable steps, which turns the fan at a designated rate of acceleration and rotation for a designated number of rotations, and then a designated rate of deceleration.

3. The apparatus for administering bilateral indirect cutaneous stimulation: the electric motor of each unit is a stepper motor having ducted fan attached to its drive shaft; and each stepper motor is activated by electrical pulses that are of sufficiently short duration so that the EDF is pulsed in short bursts.

4. The apparatus for administering bilateral indirect cutaneous stimulation to a human subject of claim 3, wherein the application of ducted air to the skin of the human subject is preferably less than one second.

5. The apparatus for administering bilateral indirect cutaneous stimulation to a human subject of claim 1, wherein the micro-controller of either unit will synchronize itself with the other based on the activation sequence; the first unit activated, or the first unit to be detected by the other, will synchronize both units. The second unit, having detected the first, follows the first.

6. An apparatus for administering bilateral nonluminous visual stimulation, said device consisting of a window; the units described in claim 1, each containing a roller fitting within the widow, one side of which is coated with a nonluminous white coating and the other with a black coating; a drive shaft on which the roller is mounted; and a brushless electric motor (composed of stator coils and a polarized magnetic rotor which divides rotation into a number of discrete steps) mounted to the roller to turn the roller; and wherein the stator coils are fed a pulsed flow of electricity, each stator coil generates a magnetic field that applies a torque to the rotor, said torque producing an opposite and equal reverse torque that turns the roller of the unit of which the motor is a part, turning the roller, which, observed by human subject viewing the window will appear as a nonluminous white apparition in the window followed by a black apparition in the window.

7. The apparatus for administering bilateral indirect cutaneous stimulation to a human subject of claim 1, and the apparatus for administering nonluminous bilateral visual stimulation to a human subject of claim 6, wherein each unit has been preprogrammed, thereby enabling the first and second unit to communicate with each other.

8. The apparatus for administering bilateral indirect cutaneous stimulation to a human subject of claim 1, and the apparatus for administering nonluminous bilateral visual stimulation to a human subject of claim 6, wherein the first and second units communicate with each other using short-wavelength UHF radio waves in the ISM band from 2.4 to 2.4835 Ghz through a coded communication protocol.

9. The apparatus for administering bilateral indirect cutaneous stimulation to a human subject of claim 1, and the apparatus for administering nonluminous bilateral visual stimulation to a human subject of claim 6, wherein the rate of bilateral tactile stimulation is a function of manual input from the human subject by way of a non-toggling switch on each unit to select from a number of preset rates indicated by a graphic interface on each unit.

10. An apparatus for administering bilateral indirect cutaneous stimulation to a human subject, said device comprising: two units with communicating microcontrollers that synchronize cutaneous stimulation events; a first cutaneous stimulation unit powered by a first rechargeable battery in contact with a right side of the human subject, said first unit containing a first micro-controller, a first EDF, a first transceiver that communicates with a second micro controller in a second unit, a first user-activated switch and a first visual interface; cutaneous stimulation unit powered by a second rechargeable battery in contact with a left side of the human subject, said second unit containing a second micro-controller, a second EDF, a second transceiver that communicates with the first micro controller, a second user-activated switch and a second graphic interface; and wherein both units are identical and consisting of an EDF forcing a flow of air through a duct and then a nozzle accelerating said flow of air against the skin of the person wearing such unit, said airflow being perceived as a cutaneous touch and/or pressure differential by the human subject.

11. The apparatus for administering bilateral indirect cutaneous stimulation to a human subject of claim 10, wherein a reverse torque that turns the fan of the unit of which the motor is a part, forcing a flow of air through the duct against the skin of the person wearing such unit, said airflow being perceived as a cutaneous touch and/or pressure differential by the human subject.

12. The apparatus for administering bilateral indirect cutaneous stimulation to a human subject of claim 10, wherein the micro-controller of either module is programmed to synchronize with the other.

13. The apparatus for administering bilateral indirect cutaneous stimulation to a human subject of claim 10, wherein a both units are programmed to wirelessly communicate with the other.

14. The apparatus for administering bilateral indirect cutaneous stimulation to a human subject of claim 13, and the apparatus for administering nonluminous bilateral visual stimulation to a human subject of claim 6, wherein the first and second units communicate with each other using short-wavelength UHF radio waves in the ISM band from 2.4 to 2.4835 Ghz through a coded communication protocol.

15. The apparatus for administering bilateral indirect cutaneous stimulation to a human subject of claim 10, and the apparatus for administering nonluminous bilateral visual stimulation to a human subject of claim 6, wherein the rate of bilateral tactile stimulation is a function of manual input from the human subject by way of a non-toggling switch on each unit to select from a number of preset rates indicated by a graphic interface on each unit.

16. An apparatus for administering bilateral indirect cutaneous stimulation to a human subject, said device comprising: two units with communicating microcontrollers that synchronize cutaneous stimulation events; a first cutaneous stimulation unit powered by a first rechargeable battery in contact with a right side of the human subject, said first unit containing a first micro-controller, a first EDF, a first transceiver that communicates with a second micro controller in a second unit, a first user-activated switch and a first visual interface; cutaneous stimulation unit powered by a second rechargeable battery in contact with a left side of the human subject, said second unit containing a second micro-controller, a second EDF, a second transceiver that communicates with the first micro controller, a second user-activated switch and a second graphic interface; and wherein both units are identical and consisting of an EDF forcing a flow of air through a duct and then a nozzle accelerating said flow of air against the skin of the person wearing such unit, said airflow being perceived as a cutaneous touch and/or pressure differential by the human subject; said units synchronizing indirect cutaneous stimulation events provided by said first and second units, said graphic interface enables a user to change the intensity, interval and duration of the indirect cutaneous stimulation events of said first and second units, turn the units on or off, and display the level of discharge of the batteries.
